(19) United States
(12) Patent Application Publication
(10) Pub. No.: US 2026/0013619 A1
(43) Pub. Date: Jan. 15, 2026

(54) MULTIFUNCTIONAL BOTTLE CAP WITH INTEGRATED PHYSIOTHERAPY AND SKINCARE TREATMENT ELEMENTS

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Chen Kunjie, Shenzhen (CN); Zhuang Weijie, Shenzhen (CN); Huang Jianlong, Shenzhen (CN)

(73) Assignee: SHENZHEN NUON MEDICAL EQUIPMENT CO., LTD., Shenzhen (CN)

(21) Appl. No.: 19/209,885

(22) Filed: May 16, 2025

(30) Foreign Application Priority Data
Jul. 15, 2024 (CN) 202421671811X
Nov. 8, 2024 (CN) 2024227308195

(51) Int. Cl. A45D 34/04; A61F 7/00; A61N 1/04; A61N 1/32; A61N 1/36; A61N 5/06; A61N 7/00

(52) U.S. Cl. CPC A45D 34/04; A61F 7/00; A61N 1/0408; A61N 1/328; A61N 1/36014; A61N 5/0616; A61N 7/00; A45D 2200/20; A61F 2007/0052; A61F 2007/0087; A61N 2005/0644; A61N 2007/0034

(57) ABSTRACT

Embodiments of the present invention provide a bottle cap with integrated therapeutic functionalities for enhanced skin care applications. The bottle cap comprises a cap body with a built-in straw for fluid extraction, a contact plate for skin interaction, and a physiotherapy component powered by a battery and controlled via a circuit board. The physiotherapy component may include vibration, thermal, or microcurrent therapy to enhance the absorption and efficacy of skincare formulations. Additionally, the system may incorporate a light source for activating photosensitive precursors and ensuring sterilization for hygienic use. A drop sensor regulates liquid dispensation, ensuring precise application. The bottle cap is designed for compatibility with various skincare formulations, offering a multifunctional, portable, and user-friendly solution that streamlines skincare routines by eliminating the need for separate beauty tools.

### TECHNICAL FIELD

The present invention relates to the field of beauty and skincare, and more particularly, to a bottle cap having integrated therapeutic functionality to enhance skincare treatments.

### BACKGROUND

With the continuous improvement of living standards, there is a growing emphasis on health and beauty, driving increased demand for advanced facial skincare technologies that enhance skin health and appearance. Traditional skincare methods primarily rely on topical products and physical therapy treatments. While skincare products such as serums, creams, and oils are widely used, they often require manual application, which limits their efficiency in delivering targeted therapeutic effects. Physical therapy devices, such as LED therapy masks and microcurrent wands, offer additional skincare benefits but are typically bulky, require separate power sources, and demand dedicated usage time.

One of the main challenges in existing skincare solutions is the lack of integration between skincare applications and physical therapy treatments. Liquid-based skincare products, such as essences and serums, are often extracted using droppers or straws and then manually applied to the skin. Meanwhile, physical therapy devices function independently, requiring users to interrupt their routine to use them separately. This separation creates an inconvenient and time-consuming process that reduces the practicality and effectiveness of beauty care routines.

Additionally, most conventional beauty devices are designed for single-function use, offering only one type of therapy, such as LED light therapy, microcurrent stimulation, or thermal therapy. As a result, users must invest in multiple devices to achieve comprehensive skincare benefits, leading to higher costs and increased complexity in their skincare regimen. Moreover, these standalone devices often require manual handling, making them less convenient for individuals with busy lifestyles.

To address these challenges, the present invention provides a bottle cap with integrated physiotherapy functionality, enabling the simultaneous application of skincare liquids and treatments. The bottle cap includes a contact plate configured to engage with the skin, a phototherapy light source for delivering light therapy, microcurrent electrodes for muscle stimulation, and a temperature control component for thermal therapy.

The present invention effectively combines skincare product application and physiotherapy treatments into a single device, improving efficiency and convenience in skincare routines. By integrating a dispensing mechanism with therapeutic functions, the invention eliminates the need for multiple separate tools, thereby reducing manual effort and enhancing treatment efficacy. The physiotherapy components operate concurrently with the application of skincare liquids, promoting enhanced absorption and maximizing skincare benefits. The multifunctional design of the bottle cap allows for seamless incorporation into daily skincare and hydration habits, providing a practical and efficient solution that enhances usability and treatment effectiveness compared to conventional skincare devices.

Given these limitations, there is a growing need for an improved makeup box that integrates an intelligently arranged display system with an efficient storage design. An optimized structure would enhance accessibility, reduce unnecessary operational steps, and streamline the user experience, making beauty routines more convenient and effective.

### OBJECTS OF THE INVENTION

Some of the objects of the invention are as follows:

An object of the present invention is to provide a bottle cap with an integrated physiotherapy function, allowing simultaneous application of skincare liquids and therapeutic treatments for enhanced efficiency and convenience.

Another object of the present invention is to provide a bottle cap with a simplified structure that combines liquid extraction and physiotherapy functionalities, eliminating the need for a separate device.

Another object of the invention is to provide a bottle cap with a contact plate that enables direct skin contact, ensuring optimal delivery of phototherapy, microcurrent stimulation, and temperature therapy.

Another object of the present invention is to enhance the efficiency of skincare absorption by integrating therapeutic functions that work in synchronization with skincare liquid application.

Another object of the present invention is to provide a bottle cap incorporating a microcurrent therapy module with electrodes to stimulate muscle activity and enhance skincare absorption.

Another object of the present invention is to provide a bottle with a precisely angled straw relative to the threaded section, ensuring efficient liquid extraction and user-friendly operation.

Another object of the present invention is to provide a bottle cap with stimulation element to activate precursor stored in the bottle.

### SUMMARY OF THE INVENTION

According to a first aspect of the present invention, a bottle cap is provided. The bottle cap comprising: a cover body configured to connect with an open end of a bottle body, the cover body having a through channel with a first end for contacting a straw body and a second end for contacting the bottle body; a contact plate attached to the cover body to form an accommodating cavity between the contact plate and the cover body, the contact plate is configured to contact skin of a user; one or more physiotherapy component installed in the accommodating cavity and configured to perform therapy on skin; and a circuit board electrically connected to the one or more physiotherapy component.

In one embodiment of the invention, the straw body is positioned at a predetermined angle relative to an axis of the second end, the predetermined angle is in a range of 3 degrees to 30 degrees.

In one embodiment of the invention, the one or more physiotherapy component comprises a phototherapy light source located in the accommodating cavity; and the contact plate comprises a light guide area, wherein the phototherapy light source is positioned to correspond to the light guide area, allowing emitted light to pass through the light guide area.

In one embodiment of the invention, the one or more physiotherapy component comprises a temperature control component connected to the contact plate and electrically connected to the circuit board, the temperature control component being configured to provide hot compress therapy, cold compress therapy, or both.

In one embodiment of the invention, the one or more physiotherapy component comprises a microcurrent therapy module, the microcurrent therapy module including: a first microcurrent electrode positioned in a first contact area on the contact plate; a second microcurrent electrode positioned in a second contact area on the contact plate; and wherein the first microcurrent electrode and second microcurrent electrodes are electrically connected to the circuit board and configured to form a closed circuit through the skin.

In one embodiment of the invention, the one or more physiotherapy components are selected from the group consisting of a phototherapy component, a vibration component, an ultrasonic component, a hot compress component, a cold compress component, a radio frequency component, and a microcurrent component.

In one embodiment of the invention, the straw includes a sensor to regulate the amount of a liquid stored or released through the straw.

In one embodiment of the invention, the bottle cap further comprises a UV emitter to sterilize, disinfect, or activate a precursor stored in the straw.

In one embodiment of the invention, the one or more physiotherapy components are integrated along the straw body to provide a therapeutic effect to the user while dispensing drops.

In one embodiment of the invention, the straw body comprises an elastic ring, a suction bulb, and a straw, wherein the elastic ring fits in the through channel of the cover body.

In one embodiment of the invention, the cover body is annular in shape and comprises a magnetic part; the bottle body has a magnetic structure to detachably connect the cover body with the bottle body.

According to a second aspect of the present invention, a bottle cap is provided. The bottle cap comprising: a cover body configured to contact with an open end of a bottle body, the cover body having a through channel with a first end for positioning a straw body and a second end for contacting to the bottle body; a contact plate configured to contact skin, the contact plate being attached to the cover body and forming an accommodating cavity between the contact plate and the cover body; one or more physiotherapy component installed in the accommodating cavity and configured to perform one or more therapy on the skin; a circuit board electrically connected to the phototherapy component; and wherein the bottle cap is configured to accommodate a dropper in a retrofit manner.

In one embodiment of the invention, the one or more physiotherapy components are selected from the group consisting of a vibration component, an ultrasonic component, a hot compress component, a cold compress component, a radio frequency component, and a microcurrent component.

In one embodiment of the invention, the straw body comprises an elastic ring, a suction bulb, and a straw, wherein the elastic ring fits in the through channel of the cover body.

In one embodiment of the invention, the straw body comprises a sealing part to seal the mouth of the bottle body.

In one embodiment of the invention, the cover body is annular in shape and comprises a magnetic part; the bottle body has a magnetic structure to detachably connect the cover body with the bottle body.

According to a third aspect of the present invention, a bottle cap is provided. The bottle cap comprising: a cover body configured to connect with an open end of a bottle body, the cover body having a through channel with a first end for installing a straw and a second end for connecting to the bottle body; an accommodating cavity in the cover body to house one or more physiotherapy component; and wherein the one or more physiotherapy component acts on the liquid in the straw to perform one or more reactions in the liquid.

In one embodiment of the invention, the one or more physiotherapy components are selected from the group consisting of a heating element, a cooling element, a UV emitter, and a phototherapy component.

In one embodiment of the invention, the UV emitter sterilizes, disinfects, or activates the liquid.

In one embodiment of the invention, the phototherapy component is used to activate a precursor stored in the straw.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In the context of the specification, the term “bottle cap” broadly refers to the assembly that facilitates both the dispensing of a liquid (eg. Skincare formulation, cosmetic) and the delivery of a skin stimulation function, typically mounted on or connected to a container bottle.

In the context of the specification, the term “Container bottle” or “bottle body” refers to any reservoir or housing structure that contains the liquid to be dispensed. It may include features such as an outlet opening for fluid communication with the liquid outlet mechanism.

In the context of the specification, the term “Shell” refers to the structural casing of the cosmetic applicator apparatus that houses internal components, defines the output end, and provides mechanical support for interfaces and features such as liquid channels, stimulation elements, or sealing covers.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 is a schematic structural diagram of the bottle cap, in accordance with an embodiment of the present invention.

FIG. 2 is a front view of the bottle cap, in accordance with an embodiment of the present invention.

FIG. 3 is a side view of the bottle cap, in accordance with an embodiment of the present invention.

FIG. 4 is a vertical cross-sectional schematic view of the bottle cap shown in FIG. 3, in accordance with an embodiment of the present invention.

FIG. 5 is a schematic diagram illustrating the assembly of the cover body and the protective cover in the bottle cap shown in FIG. 1, in accordance with an embodiment of the present invention.

FIG. 6 is a schematic diagram of the protective cover structure shown in FIG. 5, viewed from a different angle, in accordance with an embodiment of the present invention.

FIG. 7 is an exploded view of the bottle cap, in accordance with an embodiment of the present invention.

FIG. 8 is a schematic diagram illustrating the angle between the straw and the cover body in the bottle cap, in accordance with an embodiment of the present invention.

FIG. 9 is a schematic structural diagram of the bottle, in accordance with an embodiment of the present invention.

FIG. 10 is a vertical cross-sectional schematic view of the bottle shown in FIG. 9, in accordance with an embodiment of the present invention.

FIG. 11 is an exploded view of the bottle, in accordance with an embodiment of the present invention.

FIG. 12 is a schematic diagram of the cover body structure in the bottle, in accordance with an embodiment of the present invention.

FIG. 13 illustrates a bottle fitted with a bottle cover configured to fit a straw body in retrofit manner, in accordance with an embodiment of the present invention.

FIG. 14 illustrates the bottle fitted with the cover body of the bottle head with the straw body removed, in accordance with an embodiment of the present invention.

FIG. 15 illustrates the bottle fitted with the dropper while the cover body of the bottle head removed, in accordance with an embodiment of the present invention.

FIG. 16 shows the configuration of the bottle with a mouth, in accordance with an alternate embodiment of the present invention.

FIG. 17 is a cross-sectional view of the bottle cap including the bottle, the cover body, and the dropper in accordance with an embodiment of the present invention.

FIG. 18 shows an enlarged view of point A of FIG. 17, in accordance with an embodiment of the present invention.

FIG. 19 shows an enlarged view of point B of FIG. 17, in accordance with an embodiment of the present invention.

FIG. 20 shows a perspective view of the cover body of the bottle cap having the one or more physiotherapy components in accordance with an embodiment of the present invention.

FIG. 21 shows an exploded view of the cover body of the bottle head, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide a multifunctional bottle cap with integrated therapeutic functionalities, designed to enhance skincare applications by combining skincare product dispensing with physical therapy treatments. The bottle cap is structured to house a liquid extraction system, a phototherapy component, and at least one therapeutic element, such as microcurrent stimulation and temperature modulation. By integrating these features, the invention streamlines the skincare process, allowing users to apply skincare products while simultaneously receiving targeted skincare treatments.

The bottle cap includes a main cap body, which serves as the housing for the therapeutic components and liquid extraction mechanism. A straw-like dispensing tube extends from the cap body, enabling precise extraction and application of skincare liquids such as essential oils or serums. A phototherapy module is embedded within the cap structure, configured to emit specific wavelengths of light to enhance skin absorption and provide therapeutic effects. Additionally, the cap may include components such as microcurrent electrodes for muscle stimulation, heating elements for enhanced absorption, or cooling elements for soothing effects, offering a versatile skincare solution.

To ensure ease of use and ergonomic handling, the bottle cap is designed with a contact surface that interfaces directly with the skin during application. The phototherapy module is strategically positioned to deliver light therapy precisely where the skincare product is applied. The microcurrent electrodes, heating elements, or cooling components are integrated into the contact surface, ensuring effective and synchronized therapy. The internal circuitry and power supply are housed within the cap, enabling wireless operation for convenience and portability.

In an embodiment, the bottle cap further includes a protective cover to shield the therapeutic components and prevent contamination when not in use. The protective cover is designed to be easily removable or rotatable, allowing quick access to the therapy functions. Additionally, the cap structure features a user-friendly control interface, enabling users to activate or adjust therapy settings according to their skincare needs.

By integrating skincare liquid application with phototherapy and other therapeutic functionalities, the present invention eliminates the need for separate beauty tools, reducing the complexity and effort involved in skincare routines. This multifunctional bottle cap enhances the efficiency and effectiveness of skincare treatments, catering to the needs of modern users seeking convenient, high-performance beauty solutions.

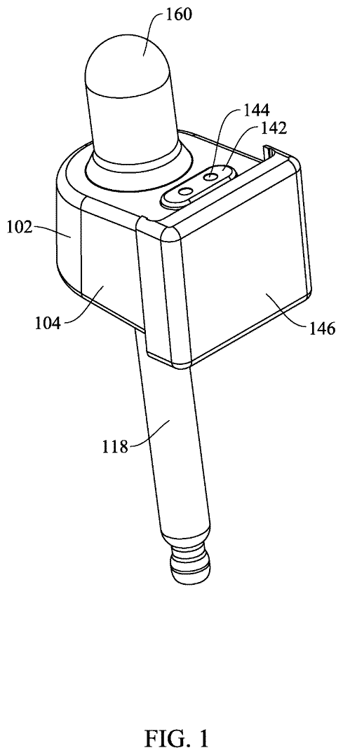

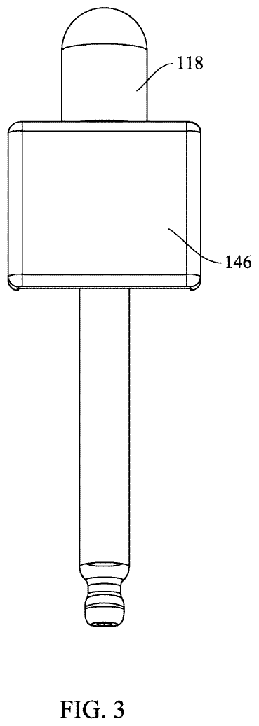

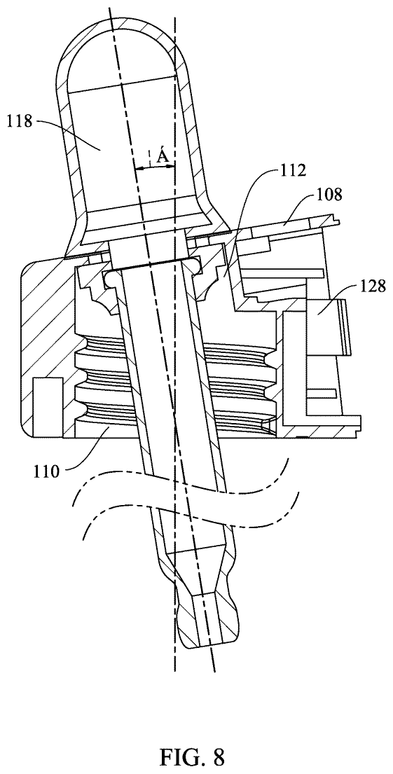

Several embodiments of the present invention will now be described in detail with references to FIGS. 1 to 12.

In an embodiment, the present invention provides a multifunctional bottle cap 102 that integrates both liquid suction and physiotherapy functionalities, enhancing skincare application efficiency.

The bottle cap 102 comprises a cap body 104, a contact plate 120, a physiotherapy component 134, a circuit board 130, and a battery 132. As illustrated in FIGS. 1 to 8, the cap body 104 is designed to securely connect with the open end of a bottle 148. A straw 118 is affixed to the cap body 104 to facilitate the extraction of liquid skincare products. The contact plate 120, mounted on the cap body 104, is configured to engage directly with the skin. An accommodating cavity 128 is formed between the contact plate 120 and the cap body 104, wherein the physiotherapy component 134 is housed and operatively coupled to the contact plate 120. The physiotherapy component 134 is designed to administer therapeutic treatment to the skin. Additionally, the circuit board 130 is positioned within the accommodating cavity 128 and electrically connected to the physiotherapy component 134 to control its operation. The battery 132, also housed in the accommodating cavity 128, provides power to the circuit board 130 and the physiotherapy component 134.

This multi-functional bottle cap 102 allows for dual functionality: first, the extraction of liquid skincare products such as essential oils through the straw 118, and second, the delivery of physiotherapeutic treatment via the physiotherapy component 134 integrated with the contact plate 120. By combining these features, users can seamlessly apply skincare products while simultaneously benefiting from targeted physiotherapy, which enhances the absorption of skincare formulations and optimizes their effectiveness. The contact plate 120, in conjunction with the physiotherapy component 134, enables efficient therapeutic care under the control of the circuit board 130 and battery 132, offering ease of use and diverse skincare functions.

As shown in FIG. 1 to FIG. 3, the straw 118 passes through both the top and bottom walls of the cap body 104, allowing efficient fluid extraction. The contact plate 120 is positioned on one side of the cap body 104. Since the physiotherapy component 134 connects to the contact plate 120, a recessed section is incorporated on that side of the cap body 104, forming the accommodating cavity 128. This cavity is designed to house the physiotherapy component 134, battery 132, and circuit board 130, ensuring a compact and organized integration. Powered by the battery 132, the physiotherapy component 134 delivers targeted therapy through the contact plate 120 to meet various skincare needs.

The physiotherapy component 134 can be selectively activated or deactivated via signals from the circuit board 130. If the physiotherapy component 134 supports multiple therapeutic functions, it can switch between different modes under circuit board control.

In an embodiment of the present invention, the circuit board is configured to connect with an external communication device over a communication interface. The control assembly includes a processor, a memory unit, and a communication interface. The communication with the external communication device is routed through the communication interface. The control assembly is configured to connect with the external communication device and receive an input. The input may correspond to activating the one or more physiotherapy components 134 or controlling one or more parameters of operating the light source assembly. The external communication device may be a smartphone, a desktop computer, a tablet, a Personal Digital Assistant (PDA), a smartwatch, or the like.

The physiotherapy component 134 may include one or more of the following: phototherapy components, vibration components, ultrasonic components, thermal therapy (hot and cold compress) components, radio frequency components, and microcurrent components.

The Phototherapy Component emits specific wavelengths of light to promote blood circulation, enhance tissue metabolism, and support skin rejuvenation. For instance, infrared light may be used to stimulate circulation and encourage cellular repair.

The Vibration Component generates mechanical vibrations to promote lymphatic circulation, reduce facial puffiness, and stimulate muscle activity, aiding in skin tightening and overall complexion enhancement.

The Ultrasonic Component utilizes ultrasound waves to induce micro-massages within the skin layers, facilitating deep tissue repair and rejuvenation.

The Thermal Therapy Component (Hot/Cold Compress) provides localized temperature therapy to the skin. The hot compress function improves blood circulation and enhances product absorption, while the cold compress function soothes inflammation and reduces swelling by constricting blood vessels.

The Radio Frequency (RF) Component emits RF waves that penetrate the dermis, stimulating collagen production and tightening the skin, which helps to reduce wrinkles and improve skin elasticity.

The Microcurrent Component applies low-level electrical currents to stimulate muscle activity, enhance metabolic function, and improve facial contouring.

For example, when the physiotherapy component 134 is configured as a vibration device, it is electrically connected to the circuit board 130 and positioned inside the contact plate 120, transmitting vibrations directly to the skin upon activation.

Similarly, when the physiotherapy component 134 functions as a phototherapy device, it consists of a phototherapy light source 136 electrically connected to the circuit board 130. The phototherapy light source 136 is embedded within or aligned with the contact plate 120, ensuring that emitted light reaches the skin surface effectively. In some embodiments, a light guide area 122 is incorporated into the contact plate 120, allowing controlled dispersion of light from the phototherapy light source 136.

In embodiments where the physiotherapy component 134 provides thermal therapy, it is configured as a temperature control module electrically connected to the circuit board 130. This module is embedded within the contact plate 120 and designed to regulate temperature, either heating or cooling the contact plate 120, to deliver the desired therapeutic effect to the skin.

For microcurrent therapy, the physiotherapy component 134 includes at least two independent electrodes (e.g., a first microcurrent electrode and a second microcurrent electrode) integrated into the contact plate 120. The controlled microcurrent flow between these electrodes stimulates the underlying skin and muscle tissue, aiding in facial contouring and skin rejuvenation.

When selecting the physiotherapy components 134 with different functions, one or more can be assembled as needed.

In the following embodiment, the physiotherapy component 134 includes a phototherapy light source 136 that provides phototherapy functionality, a temperature control component capable of delivering hot or cold compresses, and a microcurrent component that supplies microcurrents. The arrangement, distribution, and functional integration of these components within the accommodating cavity 128 are explained below.

In an alternate configuration, the physiotherapy element present in the accommodating cavity of the cover body faces the straw and acts upon the fluid present in the straw. In this particular case, the physiotherapy components are facing towards the straw. The physiotherapy component may include a UV emitter, a phototherapy component, a heating element, a cooling element, or a combination thereof. The heating element and cooling element can be used for providing heat and cold treatment to the fluid. The UV emitter is used to sterilize, disinfect, or activate a precursor in the fluid stored in a dropper. Similarly, the phototherapy component can be used to activate the fluid in the dropper.

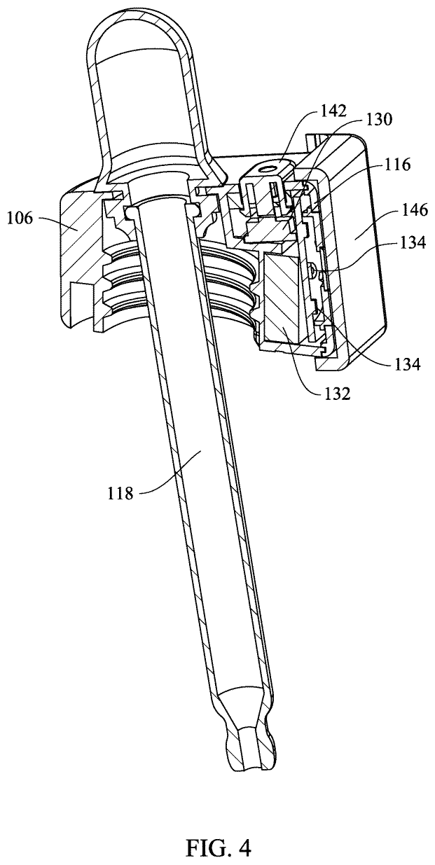

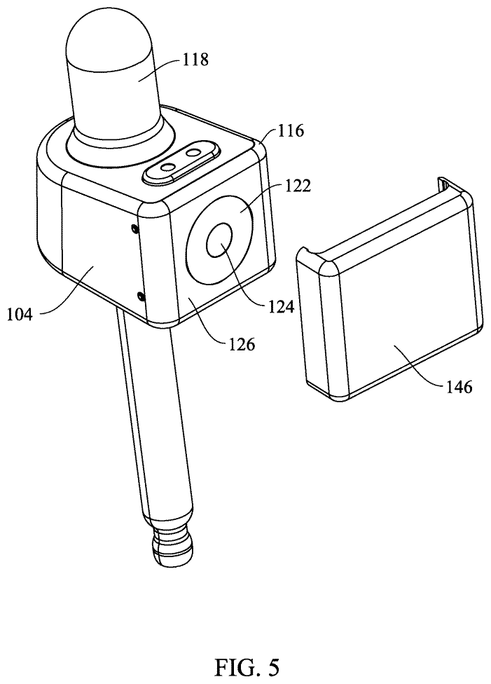

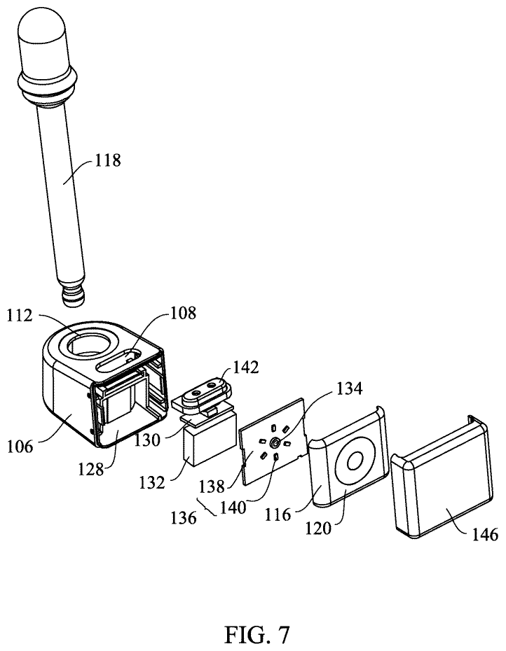

As shown in FIG. 4, FIG. 5, and FIG. 7, the physiotherapy component 134 comprises a phototherapy light source 136, a temperature control component, and a microcurrent component. The phototherapy light source 136 is positioned within the accommodating cavity 128, while the temperature control and microcurrent components are connected to the contact plate 3.

The phototherapy light source 136 consists of a lamp board 138 electrically connected to the circuit board 130 and multiple LED lamp beads 140 arranged on the lamp board 138. The temperature control component may be a cooling sheet, such as a semiconductor cooling sheet.

The LED lamp beads 140 can emit light in various wavelengths, each serving different skincare purposes. For instance: Infrared light (780 nm-1500 nm) stimulates physiological response, increase blood circulation, reduces pain, and accelerate tissue repair, Red light (620 nm-750 nm) promotes cell metabolism, stimulates collagen production, enhances skin elasticity, reduces wrinkles, and improves skin firmness, Blue light (405 nm-470 nm) exhibits antibacterial and anti-inflammatory properties, inhibits Propionibacterium acnes, and aids in acne treatment, green light (490 nm-560 nm) regulates oil secretion, soothes and calms the skin, and reduces sensitivity and redness, Yellow light (570 nm-590 nm) enhances skin immunity, brightens complexion, and improves dull skin tone.

The LED lamp beads 140 can be arranged in different combinations or used individually based on design requirements. The temperature control and microcurrent components are not illustrated in the figures.

To ensure the phototherapy light source 136 operates effectively, careful placement of the temperature control and microcurrent components within the accommodating cavity 128 is necessary. These components should not obstruct the LED lamp beads 140 from emitting light through the contact plate 120.

For optimal light transmission, the contact plate 120 includes an annular light guide area 122. Multiple LED lamp beads 140 are arranged in a circular pattern on the lamp board 138, aligning with the light guide area 122. This arrangement allows light from the LED lamp beads 140 to pass through the annular light guide area 122 and exit via the contact plate 120. Therefore, the installation of the temperature control and microcurrent components should avoid interfering with the light guide area 122.

The light guide area 122 is typically made of a transparent material such as acrylic, quartz glass, standard glass, or polycarbonate, ensuring high transparency and effective light transmission.

In alternative embodiments, the light guide area 122 may take on various shapes beyond an annular configuration, provided it separates the contact plate 120 into at least two independent sections while allowing sufficient space for the LED lamp beads 140 to function effectively.

These independent sections facilitate the operation of the microcurrent component by enabling current flow between different electrodes. They also support the temperature control component by allowing localized hot or cold compress functions.

In another embodiment, the light guide area 122 divides the contact plate 3 into a first contact area 124 and a second contact area 126. The microcurrent component is used to enable electrotherapy/TENS therapy.

In this configuration, the first contact area 124 and the second contact area 126 work in conjunction with the microcurrent component (a first microcurrent electrode and a second microcurrent electrode). Additionally, the first contact area 124 can be connected to the temperature control component to provide hot and/or cold compress functionality.

As illustrated in FIG. 5, the first contact area 124 is positioned within the light guide area 122, while the second contact area 126 is located outside the light guide area 122. The first contact area 124 is preferably composed of a metal material with high thermal and electrical conductivity, whereas the second contact area 126 is made of a metal material with good electrical conductivity. Typically, both contact areas can be fabricated from steel to ensure durability and effective performance.

Both the first contact area 124 and the second contact area 126 are electrically connected to the microcurrent component, forming a closed-loop circuit. In operation, current flows from the positive electrode, passes through the skin tissue, and returns via the negative electrode, facilitating microcurrent therapy. Additionally, the first contact area 124 is thermally coupled to the temperature control component to enable the delivery of hot or cold compress therapy.

Given that multiple functional components are housed within the accommodating cavity 128, it is essential to ensure their secure installation. In some embodiments, the LED lamp beads 140 are mounted on the lamp board 138, which is secured within the accommodating cavity 128 through mechanical constraints such as clamping or other limiting structures. This arrangement enhances the stability of the internal components and prevents displacement during use.

The substrate of the lamp board 138 is constructed from a durable material such as fiberglass-reinforced epoxy resin or an aluminum substrate. This ensures high mechanical strength, stability, and resistance to deformation under pressure or vibration.

In certain embodiments, opposing inner side walls of the accommodating cavity 128 are provided with retaining claws, as shown in FIG. 7. These claws serve to fix the lamp board 138, which houses the LED lamp beads 140, while also creating a designated space for the battery 132 within the accommodating cavity 128. During assembly, the battery 132 is first positioned inside the accommodating cavity 128, after which the lamp board 138 is secured in place adjacent to the battery 132 using the retaining claws. This configuration ensures that the LED lamp beads 140 are precisely aligned with the light guide area 122 on the contact plate 120, optimizing light emission. The placement of the temperature control component and microcurrent component can be adjusted as required.

In some embodiments, the contact plate 120 is directly affixed to the cap body 104 through snap-fit engagement or adhesive bonding. In other implementations, the cap body 104 comprises a cover body 106 and a limit frame 116, which are connected via a snap-fit mechanism. The contact plate 120 is secured to the limit frame 116 and positioned centrally within the limit frame.

The limit frame 116 may be integrally formed with the portion of the contact plate 120 corresponding to the second contact area 126. Alternatively, the limit frame 116 and contact plate 120 may be separate components, with the contact plate 120 being fixed to the limit frame 116 via adhesive bonding. The material of the limit frame 116 is typically identical to that of the cover body 106 to maintain structural consistency.

As shown in FIG. 4 and FIG. 7, in another embodiment, the cover body 106 is provided with a protruding block and a strip-shaped notch that aligns with corresponding structures on the limit frame 116. The limit frame 116 is formed with a groove and a strip-shaped protrusion, facilitating a secure snap-fit connection between the two components. When assembled, the protruding block engages with the groove, and the strip-shaped notch aligns with the strip-shaped protrusion, ensuring a stable and precise fit.

To enhance the structural integrity of the connection between the cover body 106 and the limit frame 116, additional adhesive bonding can be applied. This ensures that the adhesive fills any gaps during the snap-fit process, creating a strong and durable bond upon curing.

Since the contact plate 120 is exposed when installed on the cover body 106 through the limit frame 116, a protective structure is generally provided to shield it from unintended contact, contamination, or wear caused by external factors such as dust, moisture, or chemicals.

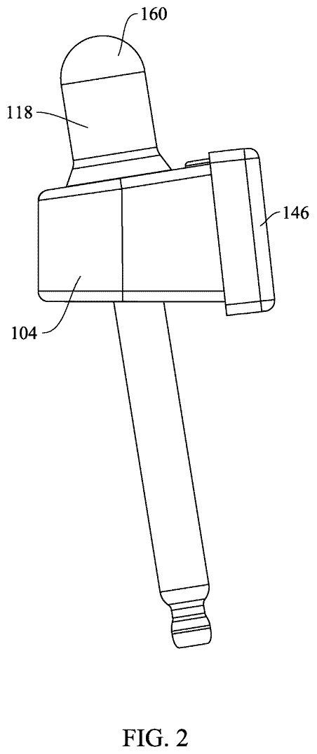

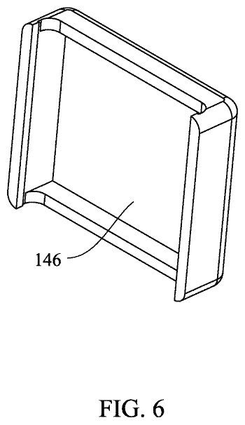

In another embodiment of the present invention, a protective cover 146 is further provided outside the limit frame 116, as shown in FIG. 2, FIG. 5, and FIG. 6.

It can be seen that the two circumferential side walls adjacent to the contact plate 120 of the cover body 106 are both provided with a plurality of flexible, fixed convex points. When the protective cover 146 is covered on the outside of the contact plate 120, the fixed convex points are squeezed and produce recoverable deformation. The protective cover 146 is initially fixed to the cover body 106 through the fixed convex points.

In order to further avoid the possibility of the protective cover 146 sliding off the cap body 104, the protective cover 146 is provided to bend one side end portion for matching with the top wall of the cap body 104 and form a bending portion that can cover the upper edge of the limit frame 116; similarly, the protective cover 146 can also form the above bending portion on one side for matching with the bottom wall of the cap body 104. The protective cover 146 with the bending portion can be better connected to the cap body 104.

The contact plate 120 functions in coordination with the physiotherapy component 134 to deliver therapeutic effects. As the physiotherapy component 134, housed within the accommodating cavity 128, operates under the control of the battery 132 and the circuit board 130, a mounting hole 108 is formed on a side of the cover body 106 adjacent to the contact plate 120 to facilitate control access. A switch member is positioned on the circuit board 130 at a location corresponding to the mounting hole 108. A pressing member 142 is installed within the mounting hole 108 and extends into the accommodating cavity 128. The pressing member 142 is configured to actuate the switch member when pressed. Specifically, the mounting hole 108 passes through the top wall of the cover body 106 and is connected to the accommodating cavity 128.

In some embodiments, the battery 132 is rechargeable. To facilitate charging the battery 132, in some embodiments, a charging electrode 144 electrically connected to the circuit board 130 is provided on the pressing member 142, and the charging electrode 144 is connected to the battery 132 through the circuit board 130 and realizes the charging function of the battery 132. The charging electrode 144 is located at one end of the pressing member 142 protruding from the cap body 104, as shown in FIG. 1.

In other embodiments, the bottle cap 102 also includes a charging interface, which can be any other charging interface known in the art, such as a Type-C interface. One end of the charging interface is connected to the circuit board 130 and the battery 132, and the other end is provided on the surface of the bottle cap 102.

Furthermore, in an additional embodiment, LEDs are integrated along the straw 118 in the over body 106 to activate a precursor stored in the bottle 148. These LEDs are strategically positioned along the suction tube to ensure optimal illumination of the liquid as it is drawn up. The LEDs are electrically connected to the circuit board 130 and are powered by the battery 132. When the suction process begins, the LEDs are activated, enabling the precursor activation at the precise moment of liquid dispensing. The wavelength of the LEDs can be adjusted based on the specific activation requirements of the precursor, such as ultraviolet (UV), infrared or visible light. To ensure durability and prevent liquid exposure, the LEDs are encapsulated within a protective layer along the straw 118 while maintaining effective illumination. When the LEDs are illuminated in the bottle cap, the light is guided through strw till bottom so that the whole of the liquid in the straw 118 gets activated.

In an embodiment of the present invention, LEDs are integrated along the straw such that when a user is using the straw for dispensing a drop on the skin of a user or the eyes of the user, the LEDs can be used to provide phototherapy on the area.

To enhance the liquid absorption efficiency of the bottle cap 102, the bottle cap incorporates a dropper-style suction mechanism, consisting of a suction bulb 160 and the straw 118. This mechanism allows for precise extraction and dispensing of liquids from a bottle 148. The suction bulb 160 is positioned at the top of the bottle cap 102, and the suction bulb 160 is made from an elastic material (such as silicon or rubber), allowing it to be manually compressed and released. When the suction bulb 160 is pressed, the air is expelled from the straw 118. Upon release, the vacuum created draws liquid from the bottle 148 into the straw 118.

Considering the functionality of the bottle cap 102, to improve liquid absorption efficiency, the through channel provided in the cap body 104 includes a connecting section 112 and a threaded section 110 connected in sequence. The connecting section 112 is used to install the straw 118, while the threaded section 110 connects to the open end of the bottle 148. The straw 118 is positioned at an angle α with respect to the axis of the threaded section 110, with an angle range of 3°-30° to enhance liquid absorption from the bottom of the bottle and reduce waste.

To further optimize dispensing control, a drop sensor is incorporated within the bottle cap 102. This sensor regulates the quantity of liquid dispensed per use, ensuring consistent dosage and minimizing product waste. The feedback from the sensor can be used to regulate the triggering of the one or more physiotherapy component.

Additionally, a UV light source is integrated within the bottle cap 102, positioned near the entry point of the straw 118. This dual-function UV light source serves two purposes: (1) Pre-activating photosensitive formulations stored in the bottle, ensuring their effectiveness upon application, and (2) Sterilizing the liquid to maintain hygienic use. The UV light is activated selectively during suction or when the bottle is tilted for dispensing, preventing unwanted exposure and preserving liquid stability.

In an embodiment of the present invention, the bottle cap 102 can be fitted to any conventional dropper in a retrofit manner. The straw of the conventional dropper is fitted inside the accommodating cavity of the cover body to pass between the connecting section and the threaded section of the bottle cap.

The bottle cap 102 can be connected to the bottle 148 via the threaded section 110 for easy storage. It can be seen from FIG. 4 that there is an angle between the axis of the bottle cap 102 (i.e., the axis of the threaded section 110) and the axis of the straw 118 (i.e., the axis of the connecting section 112), allowing the straw to efficiently absorb small amounts of liquid at the bottom of the bottle.

When the angle is too large, the straw 118 may be excessively tilted, affecting grip comfort and increasing manufacturing complexity. To balance these factors, the angle α is maintained within 3°-30°, with an optional refinement to 3°-10° in other embodiments.

The bottle cap 102 of the present invention combines liquid absorption, precision dispensing, and physiotherapy functions for improved skin care applications. This integrated design eliminates the need for users to switch between separate beauty tools, thereby simplifying the skincare routine. Additionally, the compact design enhances portability, making it suitable for travel and daily use.

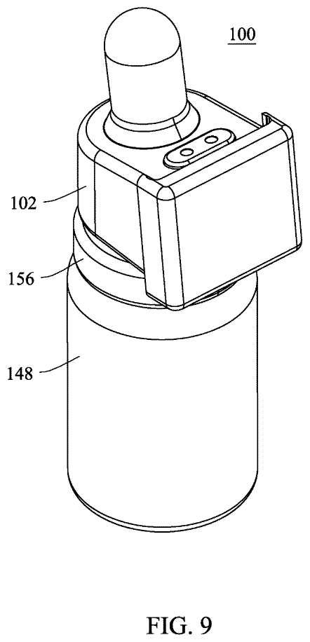

In an embodiment, the present invention a bottle assembly 100 is provided. The bottle comprising a bottle cap 102 and a bottle 148, as shown in FIG. 9 to FIG. 12. The open end of the bottle body is detachably connected to the cap body 104.

The bottle 148 can be connected to the cap body 104 of the bottle cap 102 through the open end, and the straw 118 on the bottle cap 102 can conveniently absorb the liquid in the bottle 148. The bottle 148 can not only realize the normal use and storage of the bottle cap 102 but also make it more suitable for daily use scenarios, and the use of the physiotherapy component 134 on the bottle cap 102 is more convenient.

The bottle 148 can be used not only to hold water-based skin care products but also to hold oily skin care products or skin care products of different forms of water-oil separation. It only needs to ensure that the skin care products in the bottle 148 can be sucked out by the straw 118.

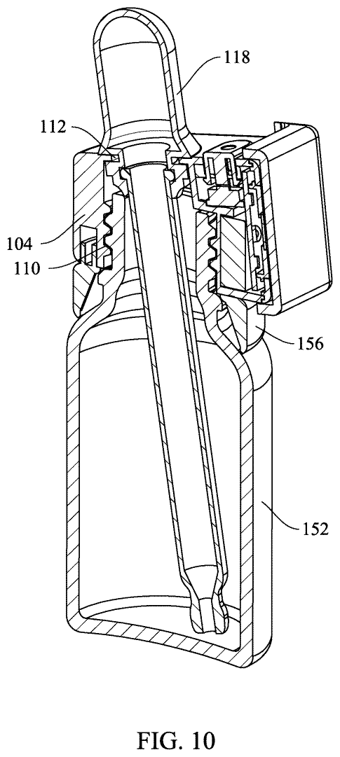

As shown in FIG. 10, it can be seen that the straw 118 is inserted obliquely relative to the bottle 148 and is adjacent to the inner bottom wall of the bottle 148.

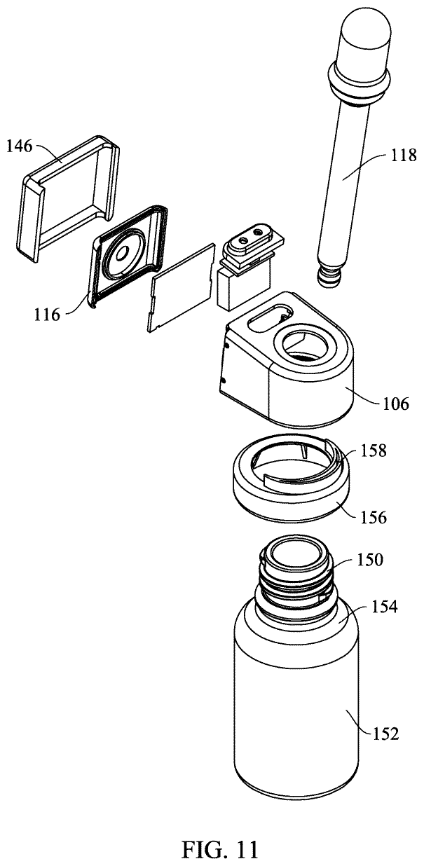

The bottleneck 150 is connected to the bottle cap 102 by a threaded connection, as shown in FIG. 10 and FIG. 11.

In another embodiment of the present invention, the bottle 148 includes a bottleneck 150, a bottle body 152, and a bottle shoulder 154, wherein the bottleneck 150 and the bottle body 152 are connected by the bottle shoulder 154. The bottleneck 150 forms the open end of the bottle 148. The outer diameter of the bottleneck 150 is smaller than the outer diameter of the bottle body 152. A connecting ring 156 is provided at one end of the bottleneck 150 near the bottle shoulder 154. The connecting ring 156 is used to cover at least part of the area of the bottle shoulder 154.

As shown in FIG. 10, the connecting ring 156 is provided on the bottle 148 adjacent to the bottle cap 102. At this time, the connecting ring 156 can be used to cover part of the area of the bottle shoulder 154 near the bottleneck 150, and part of the area of the bottle shoulder 154 near the bottle body 152 is exposed. The connecting ring 156 can play a certain sealing role at the connection between the bottleneck 150 and the bottle cap 102.

Taking the essential oil bottle as an example, the possibility of leakage and volatilization of the essential oil is reduced by filling the gap between the bottleneck 150 and the bottle cap 102, thereby ensuring the preservation quality of the essential oil.

Of course, in other embodiments, the connecting ring 156 can also be changed in external shape or color to give it a certain marking and decorative effect.

To better connect the connecting ring 156 with the bottle cap 102, in some embodiments, the connecting ring 156 is provided with a protrusion at one end thereof facing the bottleneck 150 to form a plug-in part 158, and a plug-in groove 114 matching with the plug-in part 158 is concavely provided on the bottle cap 102.

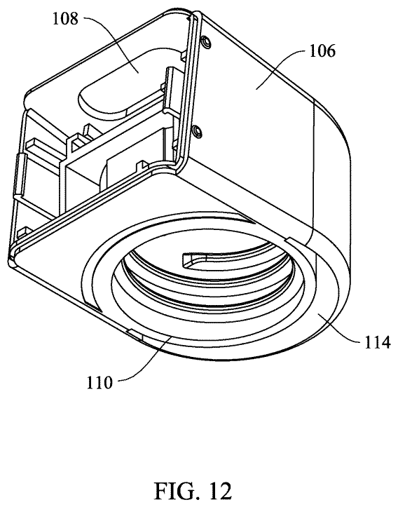

Specifically, the plug-in part 158 is approximately crescent-shaped as a whole, as shown in FIG. 11, and correspondingly, the plug-in groove 114 matching therewith can be approximately crescent-shaped, as shown in FIG. 12.

It can be understood that the bottle 148 can be matched with the bottle cap 102 to realize the storage of the bottle cap 102 and prevent the bottle cap 102 and the physiotherapy component 134 located thereon from being lost. At the same time, because the physiotherapy component 134 is combined with the bottle cap 102 and the bottle 148 by the matching contact surface, it is closely combined with daily facial care products, making the physiotherapy component 134 on the bottle cap 102 more convenient for users to use, thereby greatly improving its frequency of use and overcoming the problem of low frequency of use of traditional beauty tools.

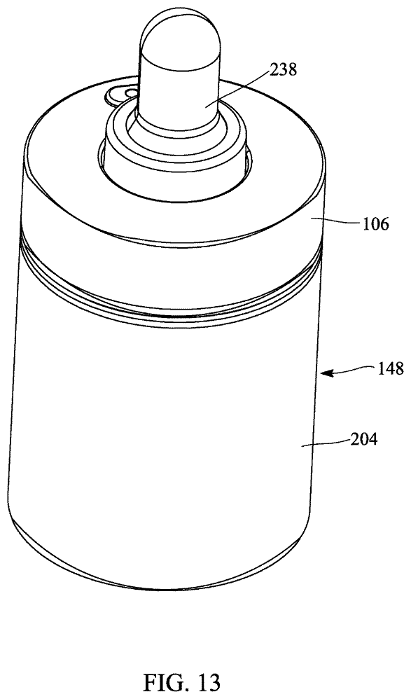

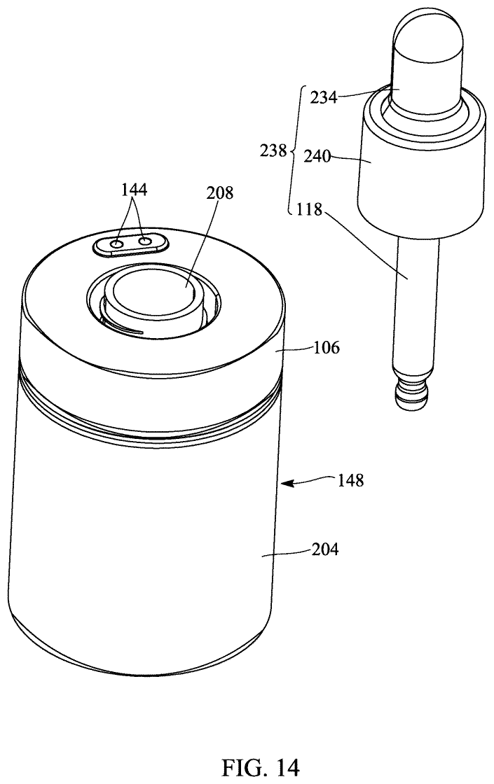

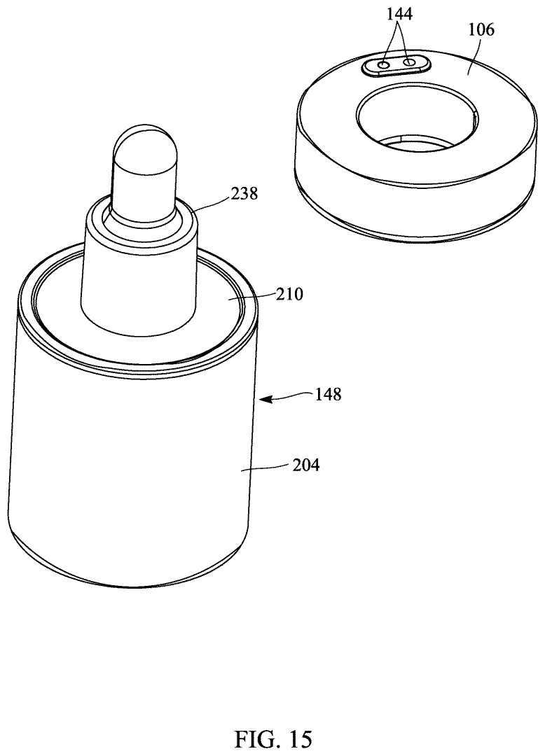

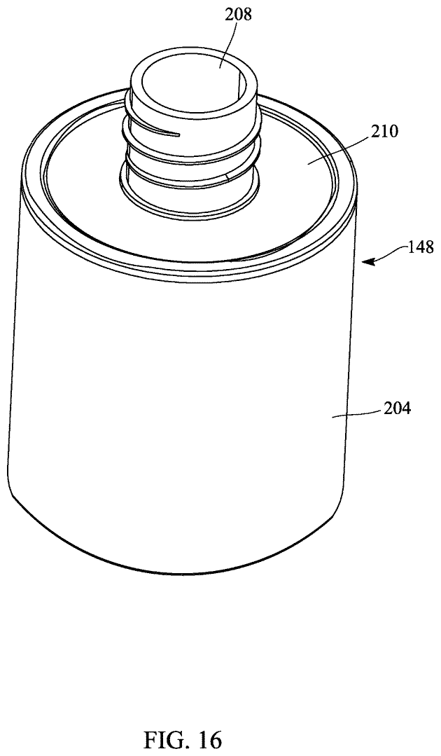

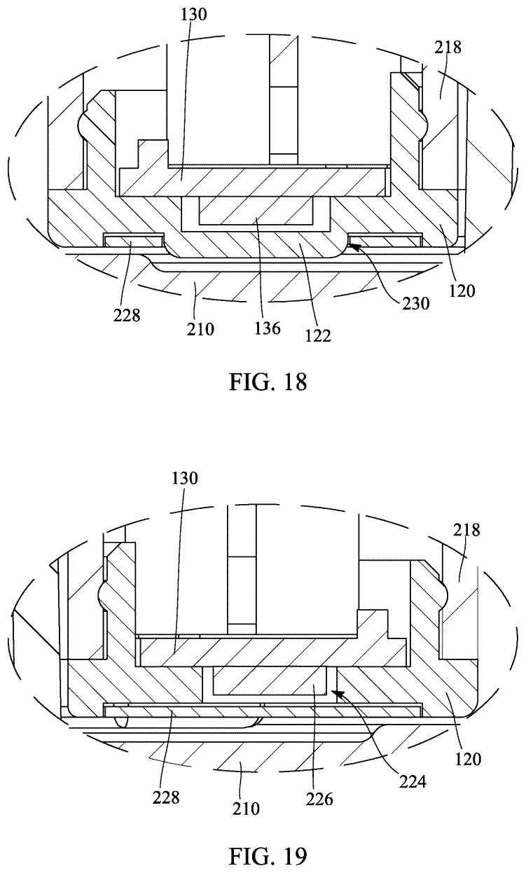

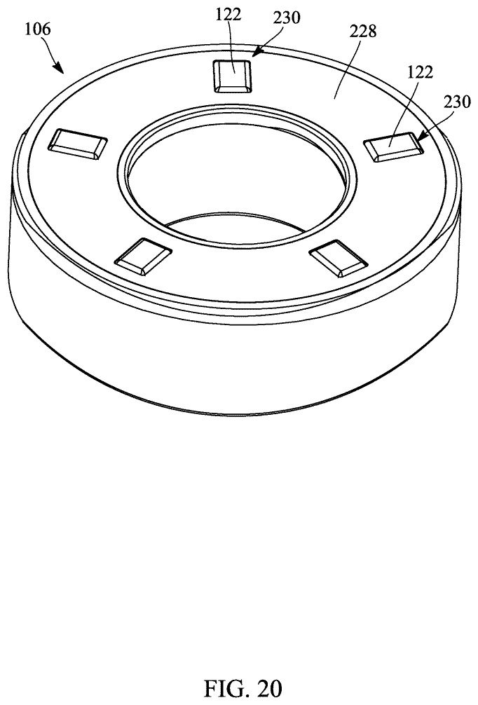

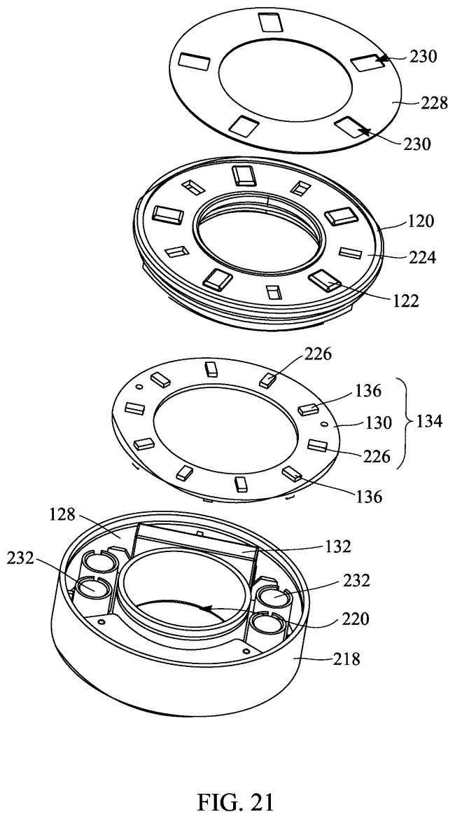

In an embodiment of the present invention, the bottle cap 102 can be fitted to any conventional dropper in a retrofit manner. The dropper is fitted inside the through channel of the cover body at the connecting section of the bottle cap. Referring to FIGS. 13 to 21, the bottle 148 and the bottle cap are shown. The bottle cap comprises a cover body 106 and a straw body 238. One end of the bottle body 152 is provided with a bottle mouth 208, onto which the straw body 238 is detachably mounted to close and seal the bottle mouth 208.

The bottle cap includes the cover body 106, and the physiotherapy component 134 is arranged in the cover body 106. The cover body 106 is sleeved over the bottle body 152 and the straw body 238, and is detachably connected to the bottle body 152.

The bottle 148 is configured to hold cosmetic substances such as essence, facial oil, essential oil, or portion. It may be implemented as a dropper bottle, spray bottle, or press pump bottle, among others. The physiotherapeutic component may include, but is not limited to, one or more of a phototherapy light source 136, a microcurrent electrode, or a hot compress mechanism.

The cover body 106 can be removed from the bottle 148 and used independently. When not in use, it may be stored by sleeving it over the bottle mouth 208. The cover body 106 may be connected to either the bottle body 152 or the bottle mouth 208. For instance, the base portion of the bottle mouth 208, adjacent to the bottle body 152, may closely fit into the through channel of the cover body 106.

In an embodiment, the straw body 238 extends outward through the through channel of the cover body 106. When the cover body 106 is installed, the straw body 238 can still be accessed and removed externally, allowing the contents of the bottle 148 to be freely dispensed through the bottle mouth 208. Thus, the cover body 106 does not obstruct access to the cosmetics.

In an embodiment, the cover body 106 is provided in an annular shape that is detachably sleeved over both the bottle body 152 and the straw body 238. This configuration allows the straw body 238 to extend outward through the through channel of the cover body 106. ensuring that cosmetics can be accessed normally even when the cover body 106 is installed. When the cover body 106 is installed on the bottle body 152, the cosmetics in the bottle body 152 can still be freely taken from the bottle mouth 208, avoiding the cover body 106 from hindering the normal use of cosmetics. At the same time, when the cover body 106 is removed from the bottle body 152 for use, it will not affect the use of cosmetics. This design enables the independent or simultaneous operation of cosmetic dispensing and physiotherapy, providing flexibility and convenience for the user based on actual usage needs.

In an embodiment, the straw body 238 is sleeved over the outer surface of the bottle mouth 208 and spaced apart from the inner wall of the cover body 106. The cover body 106 remains detachably connected to the bottle body 152. This arrangement not only improves the sealing effect at the bottle mouth 208, but also reduces the precision requirement of the fit between the cover body 106 and the bottle mouth 208, thereby facilitating the easy disassembly and reassembly of the physiotherapy component housed in the cover body 106. In alternative embodiments, the straw body 238 may also be partially inserted into the bottle mouth 208.

In an embodiment, the outer peripheral surface of the bottle mouth 208 is provided with an external thread, while the inner wall of the straw body 238 is provided with a matching internal thread, allowing the straw body 238 to be securely screwed onto the bottle mouth 208. This threaded connection ensures a reliable seal and reduces the risk of leakage. Alternatively, in other embodiments, the inner wall of the straw body 238 and the outer surface of the bottle mouth 208 may be smooth, allowing the straw body 238 to be press-fitted or sleeved over the bottle mouth 208.

In an embodiment, the straw body 238 is configured as a dropper, which is threadedly engaged with the bottle mouth 208. The inner peripheral wall of the cover body 106 is spaced apart from the straw body 238. Specifically, the straw body 238 comprises an elastic ring 240, a rubber cap 234 mounted on the elastic ring 240, and a straw 118 connected to the rubber cap 234. The elastic ring 240 is screwed onto the bottle mouth 208, and the elastic ring 240 fits into the through channel of the cover body 106, such that the cover body 106 is positioned externally around the elastic ring 240, facilitating easy access to the cosmetic contents.

In other embodiments, the straw body 238 includes a rubber plug, a rubber cap fitted onto the rubber plug, and a liquid suction tube extending from the rubber cap. The rubber plug is inserted into the bottle mouth 208 to form a seal.

In some configurations, the elastic ring 240 includes a mounting hole, and the rubber cap 234 comprises a sealing portion 236 and a suction bulb 160. The suction bulb 160 remains outside the elastic ring 240, while the sealing portion 236 is positioned inside and connected through the mounting hole. The sealing portion 236 includes a through hole for mounting the straw 118. When the elastic ring 240 is screwed onto the bottle mouth 208, the end surface of the bottle mouth abuts against the sealing portion 236, sealing the container.

In certain embodiments, a magnetic attachment mechanism is provided. A magnetic structure 214 is positioned at the end of the bottle body 152, and the cover body 106 includes a corresponding magnetic part 232. The magnetic part 232, located on the cover body 106, is magnetically coupled with the magnetic structure 214. Specifically, the magnetic part 232 is arranged on the cover body 106, and at least one of the magnetic structure 214 or the magnetic part 232 is a permanent magnet. For instance, the magnetic structure 214 may comprise an iron ring with the magnetic part 232 being a permanent magnet, or vice versa. Alternatively, both may be permanent magnets, or one may be made of ferromagnetic material. This magnetic fixation method offers advantages over interference-fit designs, such as reduced manufacturing precision requirements, less wear, and longer-lasting fixation reliability.

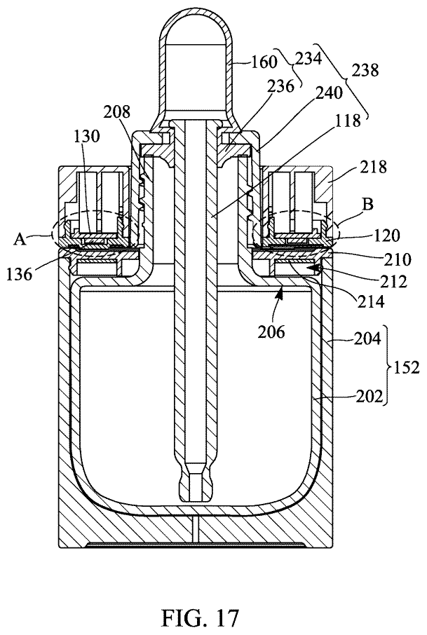

Referring to FIG. 17, the bottle body 152 includes an inner bottle 202, formed with a bottle body and a bottle mouth 208 (neck), connected by a stepped surface 206. The cover body 106 is sleeved over the bottle mouth 208. The bottle cap further includes a ring-shaped cover 210, which is also sleeved onto the bottle mouth 208 and positioned on the stepped surface 206. The ring-shaped cover 210 incorporates the magnetic structure 214. The bottle body and the bottle mouth 208 may be integrally formed from chemically stable materials like glass, while the ring-shaped cover 210 may be made of moldable materials such as plastic, facilitating integration of the magnetic components and reducing production costs.

Optionally, the ring-shaped cover 210 and the bottle mouth 208 are assembled via interference fit to ensure a tight seal.

In some embodiments, an annular mounting groove 212 is provided on the underside of the ring-shaped cover 210, facing the step surface 206 and surrounding the bottle mouth 208. The magnetic structure 214, configured as an annular magnet, is embedded within this groove. This configuration secures the magnet in place, simplifies assembly, and protects the bottle body from scratches or damage caused by direct contact with the magnetic component.

In some embodiments, the through channel in the cover body 106 is provided with a magnetic part 226 and corresponding magnetic structure is provided in the elastic ring 240 of the straw body.

In other embodiments, the bottle body 152 further includes an outer bottle 204 that is closed at one end and open at the other. The outer bottle 204 is sleeved over the inner bottle 202 and is detachably connected to the ring-shaped cover 210. Since it does not contact the cosmetic contents, the outer bottle 204 can be made from durable, easily molded materials like plastic. This design protects the inner bottle 202 from damage and allows for replacement of the inner bottle once emptied, reducing waste and cost. The outer bottle 204 may be connected to the ring-shaped cover 210 via snap-fit or interference-fit.

In some embodiments, the outer surfaces of the cover body 106, ring-shaped cover 210, and outer bottle 204 are aligned and flush, preventing the physiotherapy component 134 from protruding. This reduces the likelihood of accidental detachment during storage and enhances aesthetic and functional integration.

In alternative configurations, the inner surface of the cover body 106 may be threaded, allowing it to screw onto a bottle body 152 provided with matching external threads.

The cover body 106 may include one or more of the following: a phototherapy light source 136, a hot/cold compress module, a microcurrent stimulation module, or a vibration module. For example, in one embodiment, the phototherapy light source 136 is positioned on the end surface of the cover body 106 and emits light of specific wavelengths (e.g., red, white, blue, purple, or infrared) to perform skin therapy. This placement allows the cover body 106 to be brought close to the skin, increasing the effective treatment area and enhancing usability. In other embodiments, the lamp may be mounted along the outer circumference of the cover body 106.

Optionally, the phototherapy light source 136 is positioned at the end of the cover body 106 facing the bottle body 152, allowing it to be concealed between the shell and the bottle body when stored.

In some embodiments, the micro-current electrode and the phototherapy light source 136 are located at the same end of the cover body 106. The micro-current electrode is provided with an avoidance hole 230 at a position corresponding to the phototherapy light source 136. The arrangement of the micro-current electrode and the phototherapy light source 136 at the same end of the cover body 106 allows convenient assembling of the micro-current electrode and the phototherapy light source 136 and to provide compactness to the cover body 106.

In some embodiments, the physiotherapy component includes a heating element 226 and a heat-conducting member 228. The heating element 226 transfers heat to the heat-conducting member 228, which is exposed outside the shell for direct skin contact. When in use, the heating element 226 generates heat and conduct the heat to the heat-conducting member 228, and the heat-conducting member 228 is used to provide heat therapy to the user. The heat-conducting member 228 may also absorb body heat when the heating function is inactive, enabling cold application. The heating element 226 may include resistive wires, thermistors, or an electric heating film. The heat-conducting member 228 may be made of metal or ceramic. The heating element 226 and the heat-conducting member 228 may be directly abutted, or the heat may be transferred via a thermally conductive medium (e.g., thermal glue).

The heat-conducting member 228 may be annular and cover the end surface of the cover body 106 to increase the treatment area and improve thermal efficiency.

In some embodiments, the one or more physiotherapy components include a phototherapy light source 136 and a hot and cold compress module. The hot and cold compress module includes a heating element 226 and a heat-conducting member 228. The heat-conducting member 228 is connected to the heating element 226 through thermal conduction, and the heat-conducting member 228 is exposed on the end surface of the cover body 106. The phototherapy light source 136 is arranged close to the heat-conducting member 228 and emits light facing the end where the heat-conducting member 228 is located. The arrangement of the heat-conducting member 228 and the phototherapy light source 136 at the same end of the cover body ensures that it is convenient to centrally assemble the heat-conducting member 228 and the phototherapy light source 136, thereby improving the compactness of the structure of the cover body 106.

In some embodiments, the heat-conducting member 228 and the phototherapy light source 136 are located at the same end of the cover body 106. The heat-conducting member 228 is provided with an avoidance hole 230 at a position corresponding to the phototherapy light source 136. The arrangement of the heat-conducting member 228 and the phototherapy light source 136 at the same end of the cover body 106 allows convenient assembling of the heat-conducting member 228 and the phototherapy light source 136 and to provide compactness to the cover body 106. This configuration hides the heat-conducting member 228 and the phototherapy light source 136 between the bottle body 152 and the cover body 106 when storing, thereby providing better protection.

In some embodiments, the heating element 226 is located in the cover body 106, and the end surface of the cover body 106 has a plurality of clearance holes 224 along the circumferential direction. The heating element 226 contacts the heat-conducting member 228 through the clearance holes 224. This arrangement ensures that the heat-conducting member 228 is in full contact with the skin, thereby improving the effect of hot/cold compress, and facilitating the setting of a waterproof sealing structure to effectively protect the heating element 226.

In some embodiments, the heat-conducting member 228 is provided with an avoidance hole 230. The contact plate 120 of the cover body 106 has a light guide area 122. The light guide area 122 is protruding outward relative to the end surface of the cover body 106, and is correspondingly inserted into the avoidance hole 230. The phototherapy light source 136 is located in the accommodating cavity 128 of the cover body 106 and is correspondingly arranged with the light guide area 122. Specifically, the light guide area 122 is adapted to the avoidance hole 230, so that the positioning can be achieved by the cooperation of the light guide area 122 and the avoidance hole 230, to facilitate the positioning and installation of the heat-conducting member 228.

In some embodiments, the physiotherapy component 134 also includes a circuit board 130, on which a plurality of phototherapy light source 136 and heating elements 226 are arranged in alternating sequence along the circumference. This arrangement increases the therapeutic area while maintaining a compact structure.

The cover body 106 may comprise a main shell 218 with an annular opening 220. The main shell 218 houses the physiotherapy components in the accommodating cavity 128. The annular opening 220 acts as a through channel to allow the straw body 238 to fit into the cover body 106. The accommodating cavity 128 in main shell 218 of the cover body 106 is covered by a contact plate 120. The contact plate 120 may be covered with the heat-conducting member 228.

Power for the physiotherapy component may be supplied by the battery 132 connected to a charging electrode 144. The charging electrode may be a wireless coil, a USB Type-C port, a spring contact connector, or other types of conductive interfaces. Alternatively, a connector may be exposed on the annular shell to allow an external power supply during operation.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A bottle cap, comprising:
a cover body configured to connect with an open end of a bottle body, the cover body having a through channel with a first end for contacting a straw body and a second end for contacting the bottle body;
a contact plate attached to the cover body to form an accommodating cavity between the contact plate and the cover body, the contact plate is configured to contact skin of a user;
one or more physiotherapy components installed in the accommodating cavity and configured to perform therapy on skin; and
a circuit board electrically connected to the one or more physiotherapy components.

2. The bottle cap of claim 1, wherein the straw body is positioned at a predetermined angle relative to an axis of the second end, the predetermined angle is in a range of 3 degrees to 30 degrees.

3. The bottle cap of claim 1, wherein the one or more physiotherapy component comprises a phototherapy light source located in the accommodating cavity; and the contact plate comprises a light guide area, wherein the phototherapy light source is positioned to correspond to the light guide area, allowing emitted light to pass through the light guide area.

4. The bottle cap of claim 1, wherein the one or more physiotherapy component comprises a temperature control component connected to the contact plate and electrically connected to the circuit board, the temperature control component being configured to provide hot compress therapy, cold compress therapy, or both.

5. The bottle cap of claim 1, wherein the one or more physiotherapy components comprise a microcurrent therapy module, the microcurrent therapy module including:
a first microcurrent electrode positioned in a first contact area on the contact plate;
a second microcurrent electrode positioned in a second contact area on the contact plate; and
wherein the first microcurrent electrode and second microcurrent electrodes are electrically connected to the circuit board and configured to form a closed circuit through the skin.

6. The bottle cap of claim 1, wherein the one or more physiotherapy component is selected from the group consisting of a phototherapy component, a vibration component, an ultrasonic component, a hot compress component, a cold compress component, a radio frequency component, and a microcurrent component.

7. The bottle cap of claim 1, wherein the straw includes a sensor to regulate the amount of a liquid stored or released through the straw.

8. The bottle cap of claim 1, wherein the bottle cap further comprises a UV emitter to sterilize, disinfect, or activate a precursor stored in the straw.

9. The bottle cap of claim 1, wherein the one or more physiotherapy components are integrated along the straw body to provide a therapy effect to the user while dispensing drops.

10. The bottle cap of claim 1, wherein the straw body comprises an elastic ring, a suction bulb, and a straw, wherein the elastic ring fits in the through channel of the cover body.

11. The bottle cap of claim 1, wherein the cover body is annular in shape and comprises a magnetic part; and the bottle body has a magnetic structure to detachably connect the cover body with the bottle body.

12. A bottle cap, comprising:
a cover body configured to contact with an open end of a bottle body, the cover body having a through channel with a first end for positioning a straw body and a second end for contacting the bottle body;
a contact plate configured to contact skin, the contact plate being attached to the cover body and forming an accommodating cavity between the contact plate and the cover body;
one or more physiotherapy components installed in the accommodating cavity and configured to perform one or more therapies on the skin;
a circuit board electrically connected to the one or more physiotherapy components; and
wherein the bottle cap is configured to accommodate a dropper in a retrofit manner.

13. The bottle cap of claim 12, wherein the one or more physiotherapy component is selected from the group consisting of a vibration component, an ultrasonic component, a hot compress component, a cold compress component, a radio frequency component, and a microcurrent component.

14. The bottle cap of claim 12, wherein the straw body comprises an elastic ring, a suction bulb, and a straw, wherein the elastic ring fits in the through channel of the cover body.

15. The bottle cap of claim 12, wherein the straw body comprises a sealing part to seal a mouth of the bottle body.

16. The bottle cap of claim 12, wherein the cover body is annular in shape and comprises a magnetic part; and the bottle body has a magnetic structure to detachably connect the cover body with the bottle body.

17. A bottle cap, comprising:
a cover body configured to contact with an open end of a bottle body, the cover body having a through channel with a first end for positioning a straw body and a second end for contacting the bottle body;
an accommodating cavity in the cover body to house one or more physiotherapy components; and
wherein the one or more physiotherapy components act on a liquid in the straw to perform one or more reactions in the liquid.

18. The bottle cap of claim 17, wherein the one or more physiotherapy components are selected from the group consisting of a heating element, a cooling element, a UV emitter, and a phototherapy component.

19. The bottle cap of claim 18, wherein the UV emitter sterilizes, disinfects, or activates the liquid.

20. The bottle cap of claim 17, wherein the one or more physiotherapy components are used to activate a precursor stored in the straw.
